(19) United States
(12) Patent Application Publication
(10) Pub. No.: US 2004/0198707 A1
(43) Pub. Date: Oct. 7, 2004

(54) Means and method for hormonal contraception

(76) Inventors: Agatha Antonia Magdalena Van Beek, Uden (NL); Herman Jan Tijmen Coelingh Bennink, Driebergen (NL)

Correspondence Address: WEBB ZIESENHEIM LOGSDON ORKIN & HANSON, P.C., 700 KOPPERS BUILDING, 436 SEVENTH AVENUE, PITTSBURGH, PA, 15219, US

(21) Appl. No.: 10/478,363

(22) PCT Filed: May 23, 2002
(86) PCT No.: PCT/NL02/00329

(30) Foreign Application Priority Data
May 23, 2001 (EP) 01201947.7

(51) Int. Cl. A61K 31/56

(52) U.S. Cl. 514/170

(57) ABSTRACT

The present invention is concerned with a kit containing a plurality of hormone units for use in a contraceptive method which consists of three alternating consecutive phases, sometimes referred to as a sequential method or sequential regimen. More particularly the present invention relates to a kit containing a plurality of daily hormone units for use in a contraceptive method which consists of three consecutive phases—an estrogenic, a progestogenic and a hormone-free phase—said method comprising administering to a female of childbearing capability a) during the estrogenic phase one or more hormone units to provide a therapeutically effective amount of synthetic estrogen or a combination of synthetic estrogen and biogenic estrogen to inhibit ovulation and (b) during the progestogenic phase one or more hormone units to provide a combination of biogenic estrogen and progestogen in a therapeutically effective amount to inhibit ovulation and to transform the endometrium from a proliferative into a secretory state, wherein the progestogenic phase encompasses a period of at least 10 days, the hormone-free phase encompasses a period of at least 1 day and the three consecutive phases together encompass a period of 20-35 days.

### TECHNICAL FIELD

The present invention is concerned with a kit containing a plurality of hormone units for use in a contraceptive method which consists of three consecutive phases. The method according to the invention is sometimes referred to as a sequential method or sequential regimen. This sequential method comprises administering to a female of childbearing capability during one phase one or more hormone units containing estrogen in a therapeutically effective amount to inhibit ovulation, during another phase one or more hormone units, containing a combination of estrogen and progestogen in a therapeutically effective amount to inhibit ovulation and to transform the endometrium from a proliferative into a secretory state and a third phase during which no estrogen and no progestogen is administered.

### BACKGROUND OF THE INVENTION

Kits for use in a sequential contraceptive method are known in the art. EP-A 0 628 312 (Jenapharm) describes a method comprising one or more phases wherein one phase uses a combination of biogenic estrogen, synthetic estrogen and progestogen and the other phases may use a placebo, or a synthetic or biogenic progestogen, or a synthetic or biogenic estrogen, or a combination of biogenic estrogen, synthetic estrogen and progestogen, or a combination of synthetic estrogen and progestogen. In example 3 a regimen is described which consists of 3 phases: one phase of 6 days using a combination of biogenic estrogen and synthetic estrogen., another phase of 15 days using a combination of biogenic estrogen, synthetic estrogen and progestogen and a phase of 7 days during which no dosage units were administered.

### SUMMARY OF THE INVENTION

Currently on the market there are a number of contraceptive preparations which can be classified into two general types. The first one are known as monophasic preparations. These contain a constant amount of estrogen and progestogen. Undesired side effects with these pills depend on the balance between the estrogen and progestogen component of the pill. For example, with a relatively dominant progestogen pill, the preparation will, over time, result in a depletion of both estrogen and progesterone receptors. The result which can be expected is an understimulated or atrophic endometrium which may eventually cause either on-pill amenorrhea or breakthrough bleeding or spotting due to poor epithelialisation. On the other hand, with a relatively dominant estrogenic preparation, it is possible that prolonged use will result in endometrial growth with the development of unsupported fragile stroma and subsequent spotting or breakthrough bleeding.

Newer preparations known as triphasic preparations have varying levels of estrogen and progestogen; in most cases consisting of relatively constant levels of estrogen with a step-wise increase in progestogen throughout the cycle. This pattern of estrogen and progestogen administration results in a relatively dominant estrogenic formulation at the beginning of the package with increasing progestogenic activity toward the end of the package. Endometrial stability is believed to be better with these pills since the estrogenic activity at the beginning of the package induces both estrogen and progesterone receptors making the endometrium sensitive to the increased levels of progestogen towards the end of the package. The progestogenic activity produces denser, more stable endometrial stroma although the relatively long duration of progestogenic exposure, toward the end of the package, may still lead to decreased estrogen and progesterone receptors and activity. A significant problem with this type of preparation is the low dose of hormones at the beginning of the package which makes these pills vulnerable to drug interactions or missed pills which may lead to escape ovulation. The beginning of the package is the critical time in terms of escape ovulation since the user has just completed a 7 day drug-free interval during which follicular development may begin.

Almost all of the methods of hormonal contraception currently on the market have in common that they are based on a regimen which involves an administration-free interval of about 7 days whereby withdrawal bleeding simulating the natural menses occurs. Thus 21 day intervals of hormone administration alternate with 7 days during which no hormones are administered.

Another characteristic of these methods is the combined use of estrogen and progestogen throughout the administration regimen. So called “unopposed” administration of estrogen has been associated with endometrial proliferation in menopausal women who received estrogen replacement therapy. It is widely accepted that continuous “unopposed” estrogen therapy substantially increases the risk of endometrial cancer. In order to counteract the negative effects of unopposed estrogen therapy, adjunctive progestogen treatment is nowadays commonly applied, also in the field of hormonal contraception. Regular progestogen administration is believed to inhibit the continual estrogen stimulation of the endometrium through an anti-proliferative effect and appears to reduce the incidence of endometrial carcinoma in post-menopausal women receiving estrogen replacement therapy [Beral V., Banks E., Reeves G., Appleby P., “Use of HRT and the subsequent risk of cancer”, J. Epidemiol. Biostat. (1999), 4(3), 191-210].

The three aspects that are considered to be most important in hormonal contraception are contraceptive reliability, cycle control and minimum side-effects. It is a commonly held belief that the contraceptive reliability is critically dependent on the inhibition of ovulation by the progestogen constituent. Added thereto are the peripheral effects of the progestogen on the cervix, fallopian tubes, and endometrium. In combined ethinyl estradiol/progestogen preparations the daily progestogen dose is always significantly higher compared to the ovulation inhibiting dose of the progestogen alone. This has two major reasons. Firstly the addition of ethinyl estradiol increases the level of Sex Hormone Binding Globulin (SHBG). SHBG binds and inactivates both estrogens and progestogens. The free, non SHBG bound fraction of those steroids is biologically active. Due to this mechanism a higher progestogen dose is needed in combined preparations to achieve a sufficiently high free progestogen level. Secondly when adding an estrogen to the contraceptive regimen more progestogen is needed to counteract estrogen induced endometrial proliferation (monophasic/continuous combined pills) or transform the endometrium from proliferation to secretion (sequential pills). Although estrogen itself is commonly held to be added to contraceptive regimens to achieve acceptable vaginal bleeding pattern, estrogens also inhibit ovulation in a dose dependent way. Therefore an amount of estrogen that is sufficiently biologically active, either a biogenic or a synthetic estrogen or a combination thereof, will inhibit ovulation. However biogenic estrogens alone require such high dosages for inhibition of ovulation that side-effects prevent the use of such compounds. Combined ethinyl estradiol progestogen preparations that are taken over three weeks followed by an administration pause of 6-7 days have, hitherto, shown the greatest contraceptive reliability. Thus it is not surprising that these combined preparations have gained immense popularity.

An essential element of the sequential method according to the invention is the application in one phase of a synthetic estrogen in the absence of a progestogen, and the combined application of a biogenic estrogen and a progestogen in another phase. Thus the method according to the invention comprises two consecutive phases, wherein during one phase a therapeutically effective amount of synthetic estrogen or a combination of synthetic and biogenic estrogen is administered to inhibit ovulation and during the next phase a combination of biogenic estrogen and progestogen is administered in a therapeutically effective amount to inhibit ovulation and to transform the endometrium from a proliferative into a secretory state. The present method additionally comprises a third phase during which no estrogen and no progestogen is administered. The inclusion of such a hormone-free period in the present method offers the advantage of a more predictable withdrawal bleeding.

The hormone regimen used in the present method is compatible with natural female physiology. The menstruation cycle of females effectively consists of 2 hormonal phases, a proliferation phase regulated by estrogen and a secretion phase which is regulated by the combination of estrogen and progesterone. The present method uses a very similar pattern.

As regards side-effects the present method offers the advantage that it employs a biogenic estrogen during the part of the cycle wherein adequate reliability can be achieved without the use of a synthetic estrogen like ethinyl estradiol. Because biogenic estrogens are naturally present in the female body, side-effects do not normally occur as long as serum levels do not substantially exceed naturally occurring concentrations. With synthetic estrogens there is a (dose dependent) risk of undesirable side-effects, such as thromboembolism, fluid retention and breast pain.

In comparison to the known sequential methods such as those disclosed in EP-A 0 628 312 (Jenapharm) the present method offers the advantage that it provides maximum reliability by employing a synthetic estrogen during the estrogenic phase while minimising the disadvantageous side-effects of synthetic estrogen by employing biogenic estrogen during the progestogenic phase, which phase usually represents a significant part of the cycle. The use of only biogenic estrogen during the estrogenic phase is insufficient to adequately inhibit follicular development, thereby increasing the risk of ovulation.

### DETAILED DESCRIPTION OF THE INVENTION

One aspect of the present invention is concerned with a kit containing a plurality of daily hormone units for use in a contraceptive method, the plurality of daily hormone units consisting of:

a) one or more daily hormone units, for use during an estrogenic phase, containing a synthetic estrogen or a combination of synthetic estrogen and biogenic estrogen in an amount equivalent to 340 μg ethinyl estradiol,

b) at least 10 daily hormone units, for use during a progestogenic phase, containing biogenic estrogen in an amount equivalent to 0.5-5 mg 17beta-estradiol and progestogen in an amount equivalent to 30-750 μg levonorgestrel and, optionally,

c) 1 to 8 daily dosage units, for use during a hormone-free phase, containing no estrogen and no progestogen.

The one or more daily units containing synthetic estrogen are for use during the estrogenic phase of the method described below, the at least 10 units containing the combination of biogenic estrogen and progestogen are for use during the progestogenic phase of the same method, and the 1 to 8 daily dosage units free of progestogen and estrogen are for use during the hormone-free phase of that method. The preferred embodiments described below in relation to the contraceptive method of the invention are equally valid for the above kit, unless they relate to parameters which are only meaningful in relation to a method, i.e. procedure of contraception.

Another aspect of the invention relates to a kit containing hormone units for use in a contraceptive method that consists of three consecutive phases—an estrogenic, a progestogenic and a hormone-free phase—said method comprising administering to a female of childbearing capability

a) during the estrogenic phase one or more hormone units to provide a therapeutically effective amount of synthetic estrogen or a combination of synthetic estrogen and biogenic estrogen to inhibit ovulation and

b) during the progestogenic phase one or more hormone units to provide a combination of biogenic estrogen and progestogen in a therapeutically effective amount to inhibit ovulation and to transform the endometrium from a proliferative into a secretory state,

wherein the estrogenic phase encompasses a period of at least 1 day, the progestogenic phase encompasses a period of at least 10 days, the hormone-free phase encompasses a period of at least 1 day and the three consecutive phases together encompass a period of 20-35 days.

The term female, whenever referred to in here, relates to female mammals. Preferably the female mammal is a homo sapiens. For homo sapiens females are usually biologically capable of child bearing between the age of 12 and 55.

The hormone units according to the invention may be administered orally, parenterally, sublingually, transdermally, intravaginally, intranasally or buccally. The daily hormonal units can suitably be administered orally, transdermally or intravaginally. Methods for transdermal administration including the associated methods for manufacturing such systems are well known in the art. In this connection, reference may be had to U.S. Pat. Nos. 4,752,478, 4,685,911, 4,438,139 and 4,291,014.

For most human females the natural interval between menses is somewhere between 20 and 35 days. To mimic the natural cyclic menses pattern, it is preferred that the plurality of hormone units consists of 20 to 35 hormone units. Most preferably the plurality of daily hormone units consists of 21-28 hormone units. In case the present kit comprises units for use in the hormone-free phase, the plurality of hormone units preferably consists of 28 units.

In accordance with the invention, during the estrogenic phase, one or more hormone units are administered to provide a therapeutically effective amount of synthetic estrogen or a combination of synthetic and biogenic estrogen to inhibit ovulation. During said estrogenic phase it was found to be advantageous to administer a combination of synthetic and biogenic estrogen as this enables a further reduction of the dose of synthetic estrogen needed to achieve ovulation inhibition. In addition these units preferably do not contain progestogen as the presence of such hormone may adversely affect the bleeding pattern. Most preferably the units containing the synthetic estrogen are free of progestogen, anti-progestogen and androgen.

In the present method, during the progestogenic phase, hormone units are administered to provide a combination of biogenic estrogen and progestrogen in a therapeutically effective amount to inhibit ovulation and to transform the endometrium from a proliferative into a secretory state. As explained above, it is advantageous not to use synthetic estrogen during this phase. Hence in a preferred embodiment of the invention the units containing a combination of biogenic estrogen and progestogen do not contain a synthetic estrogen.

The administration of estrogen as the only hormonally active ingredient during the estrogenic phase does not interfere with the withdrawal bleeding which occurs after the progestogenic phase due to discontinuation (withdrawal) of the progestogen administration. Unopposed estrogen administration causes stimulation of the progesterone receptors in the endometrium, allowing progestogens to be optimally effective in transforming the endometrium in a successive phase. As a result of this, a reduced rate of intermenstrual breakthrough bleeding, compared to conventionally combined low-dose preparations, is achieved. It is preferred to employ synthetic, or a combination of synthetic and biogenic estrogen during the estrogenic phase so as to ensure an optimum level of contraceptive reliability.

During the hormone free phase of the present method no estrogen and no progestogen is administered to the female. Obviously this may simply be achieved by not administering any dosage units during said period. However, for reasons of regularity and compliance it may be advantageous to administer placebo's during the hormone-free phase. The hormone-free phase in the present method encompasses a period of at least 1 day. This means that if the present method employs once daily administration of dosage units, at least one placebo is administered following the progestogenic phase. Alternatively, in a once daily administration regimen, during at least one day no dosage unit is administered. In a preferred embodiment of the invention the hormone-free phase encompasses a period of 1-8 days, more preferably of 2-7 days.

The estrogenic phase of the present method may advantageously encompass a period of 2-14 days. The progestogenic phase preferably encompasses a period of 10-22 days. In a particularly preferred embodiment of the invention the estrogenic phase covers 3-12 days, the progestogenic phase covers 12-18 days, the hormone-free phase covers 2-7 days and the 3 phases together encompass a period of 26-30 days.

In a preferred embodiment of the invention the daily hormone units for use during the estrogenic phase contain the synthetic estrogen, or the combination of synthetic and biogenic estrogen, in an amount equivalent to 3-40 μg ethinyl estradiol, more preferably in an amount equivalent to 15-40 μg ethinyl estradiol.

The application of relatively low levels of estrogen offers the advantage that it minimises the risk of estrogenic side effects. Examples of side effects associated with the administration of estrogens are nausea, vomiting, breast tension, headache, mood disturbances, fluid retention, bloating, liver function disturbances, cholelithiasis, cholestatic icterus, pancreatitis, thromboembolism.

The daily hormone units for use during the progestogenic phase preferably contain the biogenic estrogen in an amount equivalent to 0.5-5 mg 17beta-estradiol. The invention makes it possible to apply relatively low levels of biogenic estrogen. Thus in another preferred embodiment the daily hormone units for use during the progestogenic phase contain the biogenic estrogen in an amount equivalent to 1-3 mg 17beta-estradiol.

The daily hormone units for use during the progestogenic phase may suitably contain the progestogen in an amount equivalent to 30-750 μg levonorgestrel. More preferably the maximum amount of progestogen in the daily unit is less than the equivalent of 250 μg levonorgestrel. The minimum amount of progestogen preferably exceeds the equivalent of 40 μg levonorgestrel. Most preferably the amount of progestogen in the daily unit is equivalent to 75-150 μg levonorgestrel.

In order to determine for a specific biogenic estrogen or synthetic estrogen the amounts that are equivalent to a cited amount of ethinyl estradiol or 17beta-estradiol, the method described by Allen and Doisy may suitably be used (Allen A., Doisy E. A., “An ovarian hormone. Preliminary report on its localization, extraction and partial purification, and action in test animals. JAMA (1923), 81, 819-821). Similarly, in order to determine for a given progestogen the amount equivalent to cited amount of levonorgestrel the method originally described by McPhail can be used (Mc Phail M. K. “The assay of progestin” J Physiol (1934), 83, 145-156). A more recent description of this method can be found in an article written by Overbeek G. A., de Visser J. “A new substance with progestational activity”, Acta Endocrinol (1956), 22,318-329. It is noted that the aforementioned methods will provide useful indications about the anticipated estrogen potency or progestogen potency of a particular hormone. However, to accurately determine the equivalent amounts that are referred to above, it is advisable to additionally conduct in vivo studies in human females.

For guidance the table below provides the conversion factors for a number of progestogens that may suitably be used in the method of the invention. These conversion factors may be used to calculate, for each progestogen mentioned in the table, an estimate of the amount of said progestogen which is equivalent to a given amount of levonorgestrel.

| | | | | equivalent to | |
|---|---|---|---|---|---|
| | | equivalent to 30 μg | | 750 μg | |
| | Conversionfactor | levonorgestrel | | levonorgestrel | |
| levonorgestrel | 1 | 30 | μg | 750 | μg |
| norethisterone | 7 | 210 | μg | 5.25 | mg |
| norgestimate | 1.7 | 51 | μg | 1.275 | mg |
| drospirenone | 20 | 600 | μg | 15 | mg |
| dydrogesterone | 133 | 4 | mg | 100 | mg |

The synthetic estrogen present in the kit according to the invention is preferably selected from the group consisting of: ethinyl estradiol, mestranol, quinestranol, precursors capable of liberating such an estrogen when used in the present contraceptive method and mixtures thereof. Most preferably the synthetic estrogen is ethinyl estradiol and/or a precursor capable of liberating ethinyl estradiol. The biogenic estrogen is preferably selected from the group consisting of: estradiol, estrone, estran, estriol, estetrol, conjugated equine estrogens, precursors capable of liberating such an estrogen when used in the present method and mixtures thereof. Most preferably the biogenic estrogen is estradiol and/or a precursor capable of liberating estradiol. The term “estradiol” encompasses both 17alpha-estradiol and 17beta-estradiol. Most preferably the biogenic estrogen is 17beta-estradiol or a precursor thereof.

The progestogen contained in the kit of the invention is preferably selected from the group consisting of levonorgestrel, norgestimate, norethisterone, dydrogesterone, drospirenone, 3-beta-hydroxydesogestrel, 3-keto desogestrel (=etonogestrel), 17-deacetyl norgestimate, 19-norprogesterone, acetoxypregnenolone, allylestrenol, anagestone, chlormadinone, cyproterone, demegestone, desogestrel, dienogest, dihydrogesterone, dimethisterone, ethisterone, ethynodiol diacetate, flurogestone acetate, gastrinon, gestodene, gestrinone, hydroxymethylprogesterone, hydroxyprogesterone, lynestrenol (=lynoestrenol), medrogestone, medroxyprogesterone, megestrol, melengestrol, nomegestrol, norethindrone (=norethisterone), norethynodrel, norgestrel (includes d-norgestrel and dl norgestrel), norgestrienone, normethisterone, progesterone, quingestanol, (17alpha)-17-hydroxy-11-methylene-19-norpregna-4,15-diene-20-yn-3-one, tibolone, algestone acetophenide, nestorone, promegestone, 17-hydroxyprogesterone esters, 19-nor-17hydroxyprogesterone, 17alpha-ethinyl-testosterone, 17alpha-ethinyl-19-nor-testosterone, d-17beta-acetoxy-13beta-ethyl-17alpha-ethinyl-gon-4-en-3-one oxime and precursors of these compounds.

Specific examples of progestogen precursor which may be employed in accordance with the present invention include: anagestone acetate, chlormadinone acetate, cyproterone acetate, gestodene acetate, hydroxymethylprogesterone acetate, hydroxyprogesterone acetate, hydroxyprogesterone hexanoate, hydroxyprogesterone caproate, hydroxyprogesterone enanthate, medroxyprogesterone acetate, megestrol acetate, melengestrol acetate, nomegestrol acetate, norethindrone acetate, norethisterone acetate, norethisterone enanthate, quingestanol acetate, (17alpha)-17-hydroxy-11-methylene-19-norpregna-4,15-diene-20-yn-3-one, tibolone, algestone acetophenide, nestorone, promegestone, 17-hydroxyprogesterone esters, 19-nor-17hydroxyprogesterone esters, 17alpha-ethinyl-testosterone.

Best results are obtained with the kit according to the invention when the synthetic estrogen is ethinyl estradiol or a precursor thereof, the biogenic estrogen is estradiol or a precursor thereof and the progestogen is selected from the group consisting of levonorgestrel, norgestimate, norethisterone, drospirenone, dydrogesterone and their precursors. Thus in a preferred embodiment the hormone units for use during the estrogenic phase contain ethinyl estradiol or a combination of ethinyl estradiol and estradiol and/or precursors thereof in a therapeutically effective amount to inhibit ovulation and the hormone units for use during the progestogenic phase contain a therapeutically effective amount of a combination of estradiol and/or a precursor thereof and a progestogen selected from the group consisting of levonorgestrel, norgestimate, norethisterone, drospirenone, dydrogesterone and their precursors, to inhibit ovulation and to transform the endometrium from a proliferative into a secretory state. Of the latter progestogens levonorgestrel and norgestimate are particularly preferred.

Throughout this document by precursors of an active ingredient are meant components capable of liberating the active ingredient when used in the present contraceptive method, particularly after administration e.g. as a result of metabolic conversion of the precursor substance. Particularly useful precursors of the hormones present in the kit according to the invention are substances that differ from these hormones in that the hydrogen in at least one of the hydroxyl groups in the hormone-molecule has been substituted by —CO—R, wherein R is a hydrocarbon radical comprising from 1-25 carbons.

It is to be understood that the present invention not only encompasses the use of estrogens and progestogens specifically mentioned in this application, but also metabolites of these hormones that display comparable functionality. In this context it is noted that, for instance, levonorgestrel is a metabolite of norgestimate and that estriol is a metabolite of 17beta-estradiol. Both these progestogens and estrogens have found application in contraceptive formulations and/or preparations for hormone replacement therapy.

The plurality of hormone units contained by the present kit can suitably consist of 1-18 daily units for use in the estrogenic phase and 10-27 daily units for use in the progestogenic phase. Preferably the plurality of units additionally comprises 1-8 daily units for use in the hormone-free phase, which units are free of estrogen and progestogen. Most preferably the plurality of daily hormone units comprise 2-14 units for use in the estrogenic phase, 10-22 units for use in the progestogenic phase and 1-8 units for use in the hormone-free phase The hormone units are preferably for oral administration and arranged in a fixed sequence corresponding to the intended order of administration in 2 or 3 phases. Preferably, the hormone units to be used in either the estrogenic, the progestogenic or the hormone-free phase are easily distinguishable, e.g. because they are different in colour and/or shape. Data indications may be provided on the packaging. The packaging may be a tube or box or a strip. The box may be circular, square, or otherwise shaped with the tablets being accommodated separately therein for ease of administration. Date indications may appear adjacent to each tablet corresponding with the days on which each tablet is to be taken. Some indication of the sequence in which the tablets are to be taken preferably appears on the packaging regardless of its form.

Generally speaking, the hormone units in the present kit are prepared according to conventionally known procedures in accordance with the method of administration. Thus, the active ingredients are prepared according to known methods in a pharmaceutically acceptable form for administration. These ingredients, in their required quantities are combined with the appropriate pharmaceutical carriers such as additives, vehicles and/or flavour ameliorating substances. These substances may be referred to as diluents, binders and lubricants. Gums, starches and sugars are also common terms. Typical of these types of substances or excipients are pharmaceutical grades of mannitol, lactose starch, magnesium stearate, sodium saccharin, talcum, cellulose, glucose, sucrose, magnesium carbonate and the like. The active ingredient(s) may comprise from about 0.01% by weight to about 99.99% by weight of the total formulation and the remainder comprises the pharmaceutically acceptable carrier. The percentage of active ingredient(s) may vary according to the delivery system or method of administration and is chosen in accordance with conventional methods known in the art. Thus, the active ingredients are compounded with the chosen carrier and in for example the case of a tablet form, placed in a tablet moulding apparatus to form the tablets which are subsequently packaged in accordance with the chosen regimen.

Another aspect of the present invention relates to the use of synthetic estrogen, biogenic estrogen and progestogen in the manufacture of a kit containing hormone units for use in a contraceptive method that consists of three consecutive phases—an estrogenic, a progestogenic and a hormone-free phase—said method comprising administering to a female of childbearing capability

a) during the estrogenic phase one or more hormone units to provide a therapeutically effective amount of synthetic estrogen or a combination of synthetic estrogen and biogenic estrogen to inhibit ovulation and

b) during the progestogenic phase one or more hormone units to provide a combination of biogenic estrogen and progestogen in a therapeutically effective amount to inhibit ovulation and to transform the endometrium from a proliferative into a secretory state,

wherein the progestogenic phase encompasses a period of at least 10 days, the hormone-free phase encompasses a period of at least 1 day and the three consecutive phases together encompass a period of 20-35 days.

In the following example, a specific embodiment of the present invention is set forth. This example is meant to be illustrative of the invention and is not meant to limit it in any way.

### EXAMPLE

Women participating in the study described in this example are all selected on the basis that they are users of ‘high’ dose monophasic ethinyl estradiol-containing combined oral contraceptives. The reason for applying this selection criterion is that there is a dose-relationship between follicular development and ethinyl estradiol whether given alone or in combination with a progestogen, i.e. less follicular development in the presence of the higher dose. Participation of the females in the study starts at a moment of (almost) complete suppression of follicular development. This condition is not met if a female has her first day of bleeding in a natural cycle, after a tablet-free interval, or immediately after a low-dose combined oral contraceptive.

A clinical study is conducted in 8 healthy young women who previously used a monophasic, combined oral contraceptive pill with at least 30 microgram of ethinyl estradiol. The women were administered 30 microgram/day of ethinyl estradiol during days 1-11 of the cycle, followed by a combination of 2 mg 17beta-estradiol with 150 microgram levonorgestrel during days 12-25. Placebo was administered for 3 days (i.e. days 26-28 of the cycle). The women then started the next cycle of administration of 11 days of estrogen and 14 days of the combination of biogenic estrogen combined with progestogen. Participants are followed by vaginal ultrasonography and blood sampling for endogenous hormones to assess ovarian function (ovulation inhibition).

Results show that ovarian function was suppressed to the extent of ovulation inhibition. No intermenstrual bleeding occurred.

1-15. (canceled)

16. A kit containing a plurality of daily hormone units for use in a contraceptive method, the plurality of daily hormone units consisting of:
a) one or more daily hormone units, for use during an estrogenic phase, containing a synthetic estrogen or a combination of synthetic estrogen and biogenic estrogen in an amount equivalent to 3-40 μg ethinyl estradiol; and
b) at least 10 daily hormone units, for use during a progestogenic phase, containing biogenic estrogen in an amount equivalent to 0.5-5 mg 17beta-estradiol and progestogen in an amount equivalent to 30-750 μg levonorgestrel and, optionally,
c) 1 to 8 daily dosage units, for use during a hormone-free phase, containing no estrogen and no progestogen.

17. The kit according to claim 16, wherein the plurality of hormone units consists of 20 to 35 daily hormone units.

18. The kit according to claim 16, wherein the units for use during the estrogenic phase also contain a biogenic estrogen.

19. The kit according to claim 16, wherein the units for use during the progestogenic phase do not contain a synthetic estrogen.

20. The kit according to claim 16, wherein the daily hormone units for use during the estrogenic phase contain the synthetic estrogen in an amount equivalent to 15-40 μg ethinyl estradiol.

21. The kit according to claim 16, wherein the daily hormone units for use during the progestogenic phase contain the biogenic estrogen in an amount equivalent to 1-3 mg 17beta-estradiol.

22. The kit according to claim 16, wherein the daily hormone units for use during the progestogenic phase contain the progestogen in an amount equivalent to 75-150 μg levonorgestrel.

23. The kit according to claim 16, wherein the synthetic estrogen is selected from the group consisting of ethinyl estradiol, mestranol, quinestranol, precursors capable of liberating such an estrogen when used in the present method and mixtures thereof.

24. The kit according to claim 16, wherein the biogenic estrogen is selected from the group consisting of estradiol, estrone, estran, estriol, estetrol, conjugated equine estrogens, precursors capable of liberating such an estrogen when used in the present method and mixtures thereof.

25. The kit according to claim 16, wherein the progestogen is selected from the group consisting of levonorgestrel, norgestimate, norethisterone, dydrogesterone, drospirenone, 3-beta-hydroxydesogestrel, etonogestrel, 17-deacetyl norgestimate, 19-norprogesterone, acetoxypregnenolone, allylestrenol, anagestone, chlormadinone, cyproterone, demegestone, desogestrel, dienogest, dihydrogesterone, dimethisterone, ethisterone, ethynodiol diacetate, fluorgestone acetate, gastrinon, gestodene, gestrinone, hydroxymethylprogesterone, hydroxyprogesterone, lynestrenol, medrogestone, medroxyprogesterone, megestrol, melengestrol, nomegestrol, norethindrone, norethynodrel, norgestrel, norgestrienone, normethisterone, progesterone, quingestranol, (17alpha)-17-hydroxy-11-methylene-19-norpregna-4,15-diene-20-yn-3-one, tibolone, algestone acetophenide, nestorone, promegestone, 17-hydroxyprogesterone esters, 19-nor-17hydroxyprogesterone, 17alpha-ethinyl-testosterone, 17alpha-ethinyl-19-nor-testosterone, d-17beta-acetoxy-13beta-ethyl-17alpha-ethinyl-gon-4-en-3-one oxime and precursors of these compounds.

26. The kit according to claim 16, wherein the hormone units for use during the estrogenic phase contain ethinyl estradiol or a combination of ethinyl estradiol and estradiol and/or precursors thereof in a therapeutrically effective amount to inhibit ovulation and the hormone units for use during the progestogenic phase contain a combination of estradiol and/or a precursor thereof and a progestogen selected from the group consisting of levonorgestrel, norgestimate, norethisterone, drospirenone, dydrogesterone and their precursors, in a therapeutrically effective amount to inhibit ovulation and to transform the endometrium from a proliferative into a secretory state.

27. The kit according to claim 16, wherein the plurality of daily hormone units comprise 1-18 units for use in the estrogenic phase and 10-27 units for use in the progestogenic phase.

28. A contraceptive method that uses a plurality of hormone units and consists of three consecutive phases—an estrogenic, a progestogenic and a hormone-free phase, comprising administering to a female of childbearing capability
a) during the estrogenic phase one or more hormone units to provide a therapeutically effective amount of synthetic estrogen or a combination of synthetic estrogen and biogenic estrogen to inhibit ovulation; and
b) during the progestogenic phase one or more hormone units to provide a combination of biogenic estrogen and progestogen in a therapeutically effective amount to inhibit ovulation and to transform the endometrium from a proliferative into a secretory state,
wherein the progestogenic phase encompasses a period of at least 10 days, the hormone-free phase encompasses a period of at least 1 day and the three consecutive phases together encompass a period of 20-35 days.

29. The method according to claim 28, wherein the units administered during the estrogenic phase also contain a biogenic estrogen.

30. The method according to claim 28, wherein the units administered during the progestogenic phase do not contain a synthetic estrogen.

31. The method according to claim 28, wherein the daily hormone units administered during the estrogenic phase contain the synthetic estrogen in an amount equivalent to 3-40 μg ethinyl estradiol.

32. The method according to claim 28, wherein the daily hormone units administered during the progestogenic phase contain the biogenic estrogen in an amount equivalent to 0.5-5 mg 17beta-estradiol.

33. The method according to claim 28, wherein the daily hormone units administered during the progestogenic phase contain the progestogen in an amount equivalent to 30-750 μg levonorgestrel.

34. The method according to claim 28, wherein the synthetic estrogen is selected from the group consisting of ethinyl estradiol, mestranol, quinestranol, precursors capable of liberating such an estrogen when used in the present method and mixtures thereof.

35. The method according to claim 28, wherein the biogenic estrogen is selected from the group consisting of estradiol, estrone, estran, estriol, estetrol, conjugated equine estrogens, precursors capable of liberating such an estrogen when used in the present method and mixtures thereof.

36. The method according to claim 28, wherein the progestogen is selected from the group consisting of levonorgestrel, norgestimate, norethisterone, dydrogesterone, drospirenone, 3-beta-hydroxydesogestrel, etonogestrel, 17-deacetyl norgestimate, 19-norprogesterone, acetoxypregnenolone, allylestrenol, anagestone, chlormadinone, cyproterone, demegestone, desogestrel, dienogest, dihydrogesterone, dimethisterone, ethisterone, ethynodiol diacetate, flurogestone acetate, gastrinon, gestodene, gestrinone, hydroxymethylprogesterone, hydroxyprotesterone, lynestrenol, medrogestone, medroxyprogesterone, megestrol, melengestrol, nomegestrol, norethindrone, norethynodrel, norgestrel, norgestrienone, normethisterone, progesterone, quingestanol, (17alpha)-17-hydroxy-11-methylene-19-norpregna-4,15-diene-20-yn-3-one, tibolone, algestone acetophenide, nestorone, promegestone, 17-hydroxyprogesterone esters, 19-nor-17hydroxyprogesterone, 17alpha-ethinyl-testosterone, 17alpha-ethinyl-19-nor-testosterone, d-17beta-acetoxy-13beta-ethyl-17alpha-ethinyl-gon-4-en-3-one oxime and precursors of these compounds.

37. The method according to claim 28, wherein the hormone units administered during the estrogenic phase contain ethinyl estradiol or a combination of ethinyl estradiol and estradiol and/or precursors thereof in a therapeutically effective amount to inhibit ovulation and the hormone units administered during the progestogenic phase contain a combination of estradiol and/or a precursor thereof and a progestogen selected from the group consisting of levonorgestrel, norgestimate, norethisterone, drospirenone, dydrogesterone and their precursors, in a therapeutically effective amount to inhibit ovulation and to transform the endometrium from a proliferative into a secretory state.

38. The method according to claim 28, wherein the method comprises administering 1-18 units containing synthetic estrogen or a combination of synthetic estrogen and biogenic estrogen during the estrogenic phase and 10-27 units containing a combination of biogenic estrogen and progestogen during the progestogenic phase.
